(19) United States
(12) Patent Application Publication
(10) Pub. No.: US 2024/0287116 A1
(43) Pub. Date: Aug. 29, 2024

(54) SEPARATION OF HUMAN MILK OLIGOSACCHARIDES FROM A FERMENTATION BROTH

(71) Applicant: DSM IP ASSETS B.V., HEERLEN (NL)

(72) Inventors: Nikolay KHANZHIN, Hørsholm (DK); Pierre CHASSAGNE, Hørsholm (DK)

(21) Appl. No.: 18/570,021

(22) PCT Filed: Jun. 14, 2022
(86) PCT No.: PCT/EP2022/066133

§ 371 Date: Dec. 13, 2023

(30) Foreign Application Priority Data
Jun. 15, 2021 (DK) PA 2021 00635
Jun. 15, 2021 (DK) PA 2021 00636

(51) Int. Cl. C07H 3/06; B01J 47/014; C07H 1/08

(52) U.S. Cl. CPC C07H 3/06; B01J 47/014; C07H 1/08

(57) ABSTRACT

The invention relates to a method for recovery and purification of a neutral or sialylated human milk oligosaccharide (HMO) from a fermentation broth, comprising the steps of separating the fermentation broth to form a separated HMO-containing stream and a biomass waste stream, purifying the HMO-containing stream by nanofiltration, purifying the HMO-containing stream with an acidic cation exchange resin, concentrating the purified HMO-containing stream, and drying the purified HMO-containing stream to obtain a solidified neutral or sialylated HMO. Moreover, the invention also concerns a neutral or sialylated human milk oligosaccharide obtained by the inventive method, as well as its use in food, feed, and medical application.

### FIELD OF THE INVENTION

The present invention relates to the separation and isolation of neutral or sialylated human milk oligosaccharides (HMOs) from a reaction mixture in which they are produced.

### BACKGROUND OF THE INVENTION

During the past decades, the interest in the preparation and commercialisation of human milk oligosaccharides (HMOs) has been increasing steadily. The importance of HMOs is directly linked to their unique biological activities, therefore HMOs have become important potential products for nutrition and therapeutic uses. As a result, low cost ways of producing industrially HMOs have been sought.

To date, the structures of more than 140 HMOs have been determined, and considerably more are probably present in human milk (Urashima et al.: Milk oligosaccharides, Nova Biomedical Books, 2011; Chen Adv. Carbohydr. Chem. Biochem. 72, 113 (2015)). The HMOs comprise a lactose (Galβ1-4Glc) moiety at the reducing end and may be elongated with an N-acetylglucosamine, or one or more N-acetyllactosamine moiety/moieties (Galβ1-4GlcNAc) and/or a lacto-N-biose moiety (Galβ1-3GlcNAc). Lactose and the N-acetyllactosaminylated or lacto-N-biosylated lactose derivatives may further be substituted with one or more fucose and/or sialic acid residue(s), or lactose may be substituted with an additional galactose, to give HMOs known so far.

Efforts to develop processes for synthesizing HMOs, including sialylated HMOs, have increased significantly in the last ten years due to their roles in numerous human biological processes. In this regard, processes have been developed for producing them by microbial fermentations, enzymatic processes, chemical syntheses, or combinations of these technologies.

Direct fermentative production of HMOs, especially of those being a trisaccharide, has recently become practical (Han et al. Biotechnol. Adv. 30, 1268 (2012) and references cited therein). Such fermentation technology has used a recombinant E. coli system wherein one or more types of glycosyl transferases originating from viruses or bacteria have been co-expressed to glycosylate exogenously added lactose, which has been internalized by the LacY permease of the E. coli.

However, the use of a recombinant glycosyl transferase, especially series of recombinant glycosyl transferases to produce oligosaccharides of four or more monosaccharide units, has always led to by-product formation resulting in a complex mixture of oligosaccharides in the fermentation broth. Further, a fermentation broth inevitably contains a wide range of non-oligosaccharide substances such as cells, cell fragments, proteins, protein fragments, DNA, DNA fragments, endotoxins, caramelized by-products, minerals, salts, or other charged molecules. Therefore, to isolate neutral or sialylated HMOs from a complex matrix such as a fermentation broth is a challenging task.

For separating HMOs from carbohydrate by-products and other contaminating components, active carbon treatment combined with gel filtration chromatography has been proposed as a method of choice (WO 01/04341, EP-A-2479263, Dumon et al. Glycoconj. J. 18, 465 (2001), Priem et al. Glycobiology 12, 235 (2002), Drouillard et al. Angew. Chem. Int. Ed. 45, 1778 (2006), Gebus et al. Carbohydr. Res. 361, 83 (2012), Baumgartner et al. ChemBioChem 15, 1896 (2014)). Although gel filtration chromatography is a convenient lab scale method, it cannot be efficiently scaled up for industrial production.

EP-A-2896628 describes a process for purification of 2′-FL from a fermentation broth obtained by microbial fermentation comprising the following steps: ultrafiltration, strong cation exchange resin chromatography (H⁺-form), neutralization, strong anion exchange resin chromatography (acetate-form), neutralization, active carbon treatment, electrodialysis, second strong cation exchange resin chromatography (H⁺- or Na⁺-form), second strong anion exchange resin chromatography (C⁻-form), second active carbon treatment, optional second electrodialysis and sterile filtration. Such a purification process is intrinsically limited to neutral human milk oligosaccharides.

WO 2017/182965 and WO 2017/221208 disclose a process for purification of LNT or LNnT from fermentation broth comprising ultrafiltration, nanofiltration, active carbon treatment and treatment with strong cation exchange resin (H⁺-form) followed by weak anion exchange resin (base form).

WO 2015/188834 and WO 2016/095924 disclose the crystallization of 2′-FL from a purified fermentation broth, the purification comprising ultrafiltration, nanofiltration, active carbon treatment and treatment with strong cation exchange resin (H⁺-form) followed by weakly basic resin (base form).

Other prior art documents have disclosed purification methods elaborated for low lactose or no-lactose fermentation broths. According to these procedures, lactose added in excess during the fermentative production of a neutral HMO has been hydrolysed in situ after completion of the fermentation by the action of a β-galactosidase, resulting in a broth that substantially does not contain residual lactose. Accordingly, WO 2012/112777 discloses a series of steps to purify 2′-FL comprising centrifugation, capturing the oligosaccharide on carbon followed by elution and flash chromatography on ion exchange media. WO 2015/106943 discloses purification of 2′-FL comprising ultrafiltration, strong cation exchange resin chromatography (H⁺-form), neutralization, strong anion exchange resin chromatography (C⁻-form), neutralization, nanofiltration/diafiltration, active carbon treatment, electrodialysis, optional second strong cation exchange resin chromatography (Na⁺-form), second strong anion exchange resin chromatography (Cl⁻-form), second active carbon treatment, optional second electrodialysis and sterile filtration. WO 2019/063757 discloses a process for purification of a neutral HMO comprising separating biomass from fermentation broth and treatment with a cation exchange material, an anion exchange material, and a cation exchange adsorbent resin.

Antoine et al. Angew. Chem. Int. Ed. 44, 1350 (2005) and US 2007/0020736 disclosed the production of 3′-SL and accompanying di- and trisialylated lactoses by a genetically modified E. coli; the broth containing approx. 0.8 mM 3′-SL was treated as follows: adsorption of the products from the centrifuged supernatant on charcoal/celite, washing away the water soluble salts with distilled water, eluting the compounds by gradient aqueous ethanol, separation of the sialylated products on a Biogel column and desalting, leading to 49 mg of 3′-SL from 1 litre of broth. WO 01/04341 and Priem et al. Glycobiology 12, 235 (2002) disclosed the production of 3′-SL by a genetically modified E. coli; 3′-SL was isolated by the following sequence of operations: heat permeabilization of the producing cells followed by centrifugation, adsorption of the product from the supernatant on charcoal/celite, washing away the water soluble salts with distilled water, eluting the compound by gradient aqueous ethanol, binding the compound to a strong anion exchanger in HCO₃⁻-form, elution of the compound with a linear gradient NaHCO₃-solution, then eliminating the sodium bicarbonate with a cation exchanger (in H⁺-form), resulting in isolated 3′-SL with 49% purification yield. WO 2007/101862 and Fierfort et al. J. Biotechnol. 134, 261 (2008) disclosed an alternative work-up procedure of a 3′-SL fermentation broth, the procedure comprising the steps of heat permeabilization of the producing cell, centrifugation, adjusting the pH of the extracellular to 3.0 by the addition of a strong cation exchanger resin in acid form, removal of the precipitated proteins by centrifugation, adjusting the pH of the supernatant to 6.0 by the addition of a weak anion exchanger in base form, binding the sialyllactose to an anion exchanger in HCO₃⁻-form, after washing with distilled water, elution of the compound with a continuous gradient NaHCO₃-solution, eliminating the sodium bicarbonate with a cation exchanger (in H⁺-form) until pH 3.0 was reached, then adjustment of the pH to 6.0 with NaOH. The above purification allowed to isolate 15 g of 3′-SL from 1 litre of broth containing 25.5 g of 3′-SL. Drouillard et al. Carbohydr. Res. 345, 1394 (2010)) applied Fierfort's procedure above to a fermentation broth containing 6′-SL (11 g/l) and some 6,6′-disialyllactose (DSL), and thus isolated 3.34 g 6′-SL+DSL in a ratio of 155/86.

WO 2006/034225 describes two alternative purifications of 3′-SL from a producing fermentation broth. According to the first procedure, the lysate from the culture was diluted with distilled water and stirred with activated charcoal/celite. The slurry was washed with water, then the product was eluted from the charcoal/celite with aq. ethanol. According to the second method, the culture cells were heat treated and the precipitated solids were separated from the supernatant by centrifugation. The resulting supernatant was processed through a microfilter, the permeate was passed through a 10 kDa membrane, then nanofiltered. The resulting retentate was then diafiltered to collect the final sample. Both purification methods provided 90-100 mg 3′-SL from 1 litre of fermentation broth.

Both Gilbert et al. Nature Biotechnol. 16, 769 (1998) and WO 99/31224 disclose the enzymatic production of 3′-SL starting from lactose, sialic acid, phosphoenolpyruvate, ATP and CMP using a CMP-Neu5Ac synthetase/α-2,3-sialyl transferase fusion protein extract. The product was purified by a sequence of ultrafiltration (3000 MWCO), C18 reverse phase chromatography, nanofiltration/diafiltration at pH=3 and pH=7, acidification with a strong cation exchange (H⁺) resin, neutralization with NaOH solution and active charcoal decolourization.

WO 2009/113861 discloses a process for isolating sialyllactose from defatted and protein-free milk stream, comprising contacting said milk stream with a first anion exchange resin in the free base form and having a moisture content of 30-48% so that the negatively charged minerals are bound to the resin and the sialyllactose is not, followed by a treatment with a second anion exchange resin in the free base form which is a macroporous or gel type resin and has a moisture content between 50 and 70% so that the sialyllactose is bound to the resin. In this process, the sialyllactose containing stream is rather diluted (a couple of hundreds ppm of concentration) and the sialyllactose recovery from the first resin is moderate.

WO 2017/152918 discloses a method obtaining a sialylated oligosaccharide from a fermentation broth, wherein said sialylated oligosaccharide is produced by culturing a genetically modified microorganism capable of producing said sialylated oligosaccharide from an internalized carbohydrate precursor, comprising the steps of:
i) ultrafiltration (UF),
ii) nanofiltration (NF),
iii) optional activated charcoal treatment, and
iv) treating the broth with a strong anion exchange resin and/or cation exchange resin.

EP-A-3456836 discloses a method for separating a sialylated oligosaccharide from an aqueous medium, the method comprising a treatment of an aqueous solution containing said sialylated oligosaccharide with at least two types of an ion exchange resin, one being a strong anion exchange resin in Cl⁻-form and the other being a strong cation exchange resin.

WO 2019/043029 discloses a method for purifying sialylated oligosaccharides that have been produced by microbial fermentation or in vitro biocatalysis, the method comprising the steps of
i) separating biomass from the fermentation broth,
ii) removing cations from the fermentation broth or reaction mixture,
iii) removing anionic impurities from the fermentation broth or reaction mixture, and
iv) removing compounds having a molecular weight lower than that of the sialylated oligosaccharide to be purified from the fermentation broth or reaction mixture.

WO 2019/229118 discloses a method for the purification of a sialyllactose from other carbohydrates, the sialyllactose being produced by fermentation, comprising:
a) separating the cell-mass with ultrafiltration,
b) strong cationic ion exchanger treatment followed by strong anionic ion exchanger (Cl⁻-form) treatment of the filtrate,
c) first nanofiltration,
d) second nanofiltration,
e) electrodialysis,
f) reverse osmosis,
g) active charcoal treatment,
h) sterile filtration, and
i) spray-drying.

However, alternative and/or improved procedures for isolating and purifying neutral or sialylated HMOs from non-carbohydrate components of the fermentation broth in which they have been produced, especially those suitable for industrial scale, are needed to improve the recovery yield of the neutral or sialylated HMO and/or to simplify prior art methods while the purity of the neutral or sialylated HMO is at least maintained, and preferably, improved. Moreover, such alternative purification procedures preferably lead to purified neutral or sialylated HMOs that are free of proteins and recombinant materials originating from the used recombinant microbial strains, which are thus well suited for use in food, medical food, and feed applications.

### SUMMARY OF THE INVENTION

The invention relates to a method for recovery and purification of a neutral or sialylated human milk oligosaccharide (HMO) from a fermentation broth, comprising the steps of:
I. separating the fermentation broth to form a separated HMO-containing stream and a biomass waste stream;
II. purifying the separated HMO-containing stream:
   by nanofiltration/diafiltration then with an acidic cation exchange resin treatment, or with an acidic cation exchange resin treatment then by nanofiltration/diafiltration;
III. optionally concentrating the purified HMO-containing stream; and
IV. drying the purified HMO-containing stream to obtain a solidified neutral or sialylated HMO.

In another aspect, the invention relates to a neutral or sialylated human milk oligosaccharide obtained by the method according to the invention.

Another aspect of the invention relates to a neutral or sialylated human milk oligosaccharide obtained by the method according to the invention for use in medicine.

Another aspect of the invention relates to the use of a neutral or sialylated human milk oligosaccharide obtained by the method according to the invention for food and/or feed applications.

Another aspect of the invention relates to a food or cosmetic product comprising the neutral or sialylated human milk oligosaccharide obtained by the method according to the invention.

### DETAILED DESCRIPTION OF THE INVENTION

### 1. Terms and Definitions

The term “fermentation broth”, as used in this specification, refers to a product obtained from fermentation of the microbial organism. Thus, the fermentation product comprises cells (biomass), the fermentation medium, salts, residual substrate material, and any molecules/by-products produced during fermentation, such as the desired neutral or sialylated HMOs. After each step of the purification method, one or more of the components of the fermentation product is removed, resulting in more purified neutral or sialylated HMOs.

The term “monosaccharide” means a sugar of 5-9 carbon atoms that is an aldose (e.g. D-glucose, D-galactose, D-mannose, D-ribose, D-arabinose, L-arabinose, D-xylose, etc.), a ketose (e.g. D-fructose, D-sorbose, D-tagatose, etc.), a deoxysugar (e.g. L-rhamnose, L-fucose, etc.), a deoxy-aminosugar (e.g. N-acetylglucosamine, N-acetylmannosamine, N-acetylgalactosamine, etc.), a uronic acid, a ketoaldonic acid (e.g. sialic acid) or equivalents.

The term “disaccharide” means a carbohydrate consisting of two monosaccharide units linked to each other by an interglycosidic linkage.

The term “tri- or higher oligosaccharide” means a sugar polymer consisting of at least three, preferably from three to eight, more preferably from three to six, monosaccharide units (vide supra). The oligosaccharide can have a linear or branched structure containing monosaccharide units that are linked to each other by interglycosidic linkages.

The term “human milk oligosaccharide” or “HMO” means a complex carbohydrate found in human breast milk (Urashima et al.: Milk Oligosaccharides, Nova Medical Books, N Y, 2011; Chen Adv. Carbohydr. Chem. Biochem. 72, 113 (2015)). The HMOs have a core structure being a lactose unit at the reducing end that is elongated i) by a β-N-acetyl-glucosaminyl group or ii) by one or more β-N-acetyl-lactosaminyl and/or one or more β-lacto-N-biosyl units, and which core structures can be substituted by an α-L-fucopyranosyl and/or an α-N-acetyl-neuraminyl (sialyl) moiety. In this regard, the non-acidic (or neutral) HMOs are devoid of a sialyl residue, and the acidic HMOs have at least one sialyl residue in their structure. The non-acidic (or neutral) HMOs can be fucosylated or non-fucosylated. Examples of such neutral non-fucosylated HMOs include lacto-N-triose II (LNTri, GlcNAc(β1-3)Gal(β1-4)Glc), lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH). Examples of neutral fucosylated HMOs include 2′-fucosyllactose (2′-FL), lacto-N-fucopentaose I (LNFP-I), lacto-N-difucohexaose I (LNDFH-I), 3-fucosyllactose (3-FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II) and fucosyl-lacto-N-neohexaose (FLNnH). Examples of acidic HMOs include 3′-sialyllactose (3′-SL), 6′-sialyllactose (6′-SL), 3-fucosyl-3′-sialyllactose (FSL), LST a, fucosyl-LST a (FLST a), LST b, fucosyl-LST b (FLST b), LST c, fucosyl-LST c (FLST c), sialyl-LNH (SLNH), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT).

The term “sialyl” or “sialyl moiety” means the glycosyl residue of sialic acid (N-acetyl-neuraminic acid, Neu5Ac), preferably linked with α-linkage:

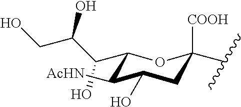

The term “fucosyl” means an L-fucopyranosyl group, preferably linked with α-interglycosidic linkage:

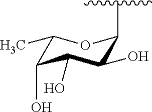

“N-acetyl-glucosaminyl” means an N-acetyl-2-amino-2-deoxy-D-glucopyranosyl (GlcNAc) group, preferably linked with β-linkage:

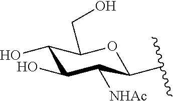

“N-acetyl-lactosaminyl” means the glycosyl residue of N-acetyl-lactosamine (LacNAc, Galpβ1-4GlcNAc), preferably linked with β-linkage:

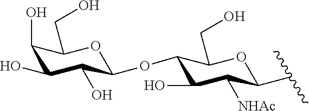

Furthermore, the term “lacto-N-biosyl” means the glycosyl residue of lacto-N-biose (LNB, Galpβ1-3GlcNAc), preferably linked with β-linkage:

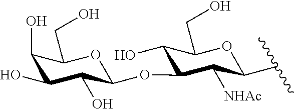

The term “biomass”, in the context of fermentation, refers to the suspended, precipitated, or insoluble materials originating from fermentation cells, like intact cells, disrupted cells, cell fragments, proteins, protein fragments, polysaccharides.

The term “Brix” refers to degrees Brix, that is the sugar content of an aqueous solution (g of sugar in 100 g of solution). In this regard, Brix of the human milk oligosaccharide solution of this application refers to the overall carbohydrate content of the solution including the human milk oligosaccharides and its accompanying carbohydrates. Brix is measured by a calibrated refractometer.

“Demineralization” preferably means a process of removing minerals or mineral salts from a liquid. In the context of the present invention, demineralization can occur in the nanofiltration step, especially when it is combined with diafiltration, or by using cation and anion exchange resins (if applicable).

The term “protein-free aqueous medium” preferably means an aqueous medium or broth from a fermentation or enzymatic process, which has been treated to remove substantially all the proteins, as well as peptides, peptide fragments, RNAs and DNAs, as well as endotoxins and glycolipids that could interfere with the eventual purification of the one or more neutral or sialylated HMOs and/or one or more of their components, especially the mixture thereof, from the fermentation or enzymatic process mixture.

The term “HMO-containing stream” means an aqueous medium containing neutral or sialylated HMOs obtained from a fermentation process, which has been treated to remove suspended particulates and contaminants from the process, particularly cells, cell components, insoluble metabolites and debris that could interfere with the eventual purification of the one or more hydrophilic oligosaccharides, especially one or more neutral or sialylated HMOs and/or one or more HMO components, especially mixtures thereof.

The term “biomass waste stream” preferably means suspended particulates and contaminants from the fermentation process, particularly cells, cell components, insoluble metabolites, and debris.

Rejection factor of a salt (in percent) is calculated as (1-κₚ/κᵣ)·100, wherein κₚ is the conductivity of the salt in the permeate and κᵣ is the conductivity of the salt in the retentate.

Rejection factor of a carbohydrate (in percent) is calculated as (1-Cₚ/Cᵣ)·100, wherein Cₚ is the concentration of the carbohydrate in the permeate and Cᵣ is the concentration of the carbohydrate in the retentate.

The term “diafiltration” refers to solvent addition (water) during the membrane filtration process. If diafiltration is applied during ultrafiltration, it improves the yield of the desired HMO in the permeate. If diafiltration is applied during nanofiltration, it improves the separation of small size impurities and salts to the permeate. The solute yield and therefore the product enrichment could be calculated based on the formulas known to the skilled person based on rejection factors and relative amount of water added.

The term “concentrating” as used in step III) of the method according to the invention refers to the removal of liquid, mostly water, thus resulting in a higher concentration of the neutral or sialylated HMO in the purified HMO-containing product stream.

### 2. Method for the Purification of a Neutral or Sialylated Human Milk Oligosaccharides from a Fermentation Broth

The invention relates to a method for recovery and purification of a neutral or sialylated human milk oligosaccharide (HMO) from a fermentation broth, comprising the steps of:
I. separating the fermentation broth to form a separated HMO-containing stream and a biomass waste stream;
II. purifying the separated HMO-containing stream by nanofiltration (NF) or nanofiltration/diafiltration (NF/DF) then with an acidic cation exchange resin treatment, or with an acidic cation exchange resin treatment then by NF/DF;
III. optionally concentrating the purified HMO-containing stream; and
IV. drying the purified HMO-containing stream to obtain a solidified neutral or sialylated HMO.

Preferably, when step II) comprises an acidic cation exchange resin treatment then NF/DF, the pH of the resin eluate is set with NaOH-solution below 5.0, preferably below 4.5, advantageously below 4.0, but preferably not less than 3.0 before performing the NF/DF step.

In a preferred embodiment, the NF/DF is conducted at a pH of less than 5.0, preferably less than 4.5, advantageously less than 4.0, but not less than 3.0.

In a preferred embodiment, step II) comprises two NF/DF steps, more preferably the second NF/DF step is conducted at a pH of less than 5.0, preferably less than 4.5, advantageously less than 4.0, but not less than 3.0.

In a preferred embodiment, step II) comprises two NF/DF steps, wherein an acidic cation exchange resin purification step is performed between the NF/DF steps, more preferably wherein the second NF/DF step is conducted at a pH of less than 5.0, preferably less than 4.5, advantageously less than 4.0, but not less than 3.0.

In this regard, in a preferred embodiment, step II) of the method comprises:
IIa. purifying the separated HMO-containing stream by nanofiltration (NF) or nanofiltration/diafiltration (NF/DF); and
IIb. an acidic cation exchange resin treatment then purification by a nanofiltration step, preferably combined with diafiltration, wherein the nanofiltration membrane has a molecular weight cut-off (MWCO) of 500-3000 Da, the active (top) layer of the membrane is composed of polyamide, the membrane has a MgSO₄ rejection factor of about 50-90% and a NaCl rejection factor of not more than 50%, and the nanofiltration step is performed so that the pH is set below 5.0, preferably below 4.5, advantageously below 4.0, but preferably not less than 3.0.

In a further preferred embodiment, the method of the invention comprises the steps of
I. separating the fermentation broth to form a separated HMO-containing stream and a biomass waste stream;
IIa. purifying the separated HMO-containing stream by nanofiltration (NF) or nanofiltration/diafiltration (NF/DF);
IIb. an acidic cation exchange resin treatment then purification by a nanofiltration step, preferably combined with diafiltration, wherein the nanofiltration membrane has a molecular weight cut-off (MWCO) of 500-3000 Da, the active (top) layer of the membrane is composed of polyamide, the membrane has a MgSO₄ rejection factor of about 50-90% and a NaCl rejection factor of not more than 50%, and the nanofiltration step is performed so that the pH is set below 5.0, preferably below 4.5, advantageously below 4.0, but preferably not less than 3.0;
III. optionally concentrating the purified HMO-containing stream; and
IV. drying the purified HMO-containing stream to obtain a solidified neutral or sialylated HMO,

with optional active charcoal treatment.

Preferably, step IIb) above comprises the addition of NaOH-solution to the acidic resin eluate so that the pH is set to 3-5 before the nanofiltration step.

In a preferred embodiment, the method does not contain a basic anion exchanger treatment step.

In a preferred embodiment, a basic anion exchanger treatment step is excluded from the method according to the invention.

In preferred embodiments, the method according to the present invention does not include an electrodialysis step and a basic anion exchange resin treatment step.

In one embodiment, the method according to the invention consists of steps I), IIa), IIb), III) and IV).

In a preferred embodiment, method steps I), IIa), IIb), III) and IV) are performed in the consecutive order I), IIa), IIb), III) and IV) as given above.

### The Fermentation Broth

In an embodiment, the neutral or sialylated HMO being present in the fermentation broth has been obtained by culturing a genetically modified microorganism capable of producing said neutral or sialylated human milk oligosaccharide from an internalized carbohydrate precursor.

Preferably, the microbial organism is a genetically modified bacterium or yeast such as a Saccharomyces strain, a Candida strain, a Hansenula strain, a Kluyveromyces strain, a Pichia strain, a Schizosaccharomyces stain, a Schwanniomyces strain, a Torulaspora strain, a Yarrowia strain, or a Zygosaccharomyces strain. More preferably, the yeast is Saccharomyces cerevisiae, Hansenula polymorpha, Kluyveromyces lactis, Kluyveromyces marxianus, Pichia pastoris, Pichia methanolica, Pichia stipites, Candida boidinii, Schizosaccharomyces pombe, Schwanniomyces occidentalis, Torulaspora delbrueckii, Yarrowia lipolytica, Zygosaccharomyces rouxii, or Zygosaccharomyces bailii; and the Bacillus is Bacillus amyloliquefaciens, Bacillus licheniformis or Bacillus subtilis.

In an embodiment, at least one neutral or sialylated human milk oligosaccharide being present in the fermentation broth has not been obtained by microbial fermentation, but has been e.g. added to the fermentation broth after it has been produced by a non-microbial method, e.g. chemical and/or enzymatic synthesis.

In an embodiment, the purity of the neutral or sialylated HMO in the fermentation broth is ≤70%, preferably ≤60%, more preferably ≤50%, most preferably ≤40%.

Preferably, the HMO is a neutral HMO. In an embodiment, the neutral HMO is preferably selected from the group consisting of 2′-fucosyllactose, 3-fucosyllactose, 2′,3-difucosyllactose, lacto-N-triose II, lacto-N-tetraose, lacto-N-neotetraose, lacto-N-fucopentaose I, lacto-N-fucopentaose II, lacto-N-fucopentaose III, lacto-N-fucopentaose V, lacto-N-neofucopentaose V (alternative name: lacto-N-fucopentaose VI), lacto-N-difucohexaose I, lacto-N-difucohexaose II, lacto-N-difucohexaose III, 6′-galactosyllactose, 3′-galactosyllactose, lacto-N-hexaose, lacto-N-neohexaose, and any mixture thereof. More preferably, the HMO is 2′-fucosyllactose, 3-fucosyllactose, 2′,3-difucosyllactose, lacto-N-triose II, lacto-N-tetraose, lacto-N-neotetraose or a lacto-N-fucopentaose, more preferably 2′-fucosyllactose, LNT, LNnT or a lacto-N-fucopentaose.

In an embodiment, the sialylated HMO is selected from the group consisting of 3′-sialyllactose (3′-SL) and 6′-sialyllactose (6′-SL).

In an embodiment, the HMO in the fermentation broth is a single neutral or sialylated HMO.

In an embodiment, the HMO in the fermentation broth is a mixture of various individual neutral or sialylated HMOs.

In an embodiment, the HMO is a mixture of two individual neutral or sialylated HMOs. In another embodiment, the HMO is a mixture of three individual neutral or sialylated HMOs. In another embodiment, the HMO is a mixture of four individual neutral or sialylated HMOs. In another embodiment, the HMO is a mixture of five individual neutral or sialylated HMOs.

In an embodiment, the HMO in the fermentation broth is a mixture of a neutral or sialylated HMO obtained by microbial fermentation and a HMO that has not been obtained by microbial fermentation, but e.g. by chemical and/or enzymatic synthesis.

### Separating the Fermentation Broth to Form a Separated HMO-Containing Stream and a Biomass Waste Stream in Step I) of the Method According to the Invention

In step I) of the method according to the invention, the HMO-containing stream is separated from the biomass waste stream.

The fermentation broth typically contains, besides the desired neutral or sialylated HMO, the biomass of the cells of the used microorganism together with proteins, protein fragments, peptides, DNAs, RNAs, endotoxins, biogenic amines, amino acids, organic acids, inorganic salts, unreacted carbohydrate acceptors such as lactose, sugar-like by-products, monosaccharides, colorizing bodies, etc. In step I) of the method according to the invention, the biomass is separated from the neutral or sialylated HMO.

In a preferred embodiment, the biomass is separated from the neutral or sialylated HMO in step I) by ultrafiltration. The ultrafiltration step is to separate the biomass and, preferably, also high molecular weight components and suspended solids from the lower molecular weight soluble components of the broth, which pass through the ultrafiltration membrane in the permeate. This ultrafiltration permeate is an aqueous solution containing the neutral or sialylated human milk oligosaccharide also referred to as the HMO-containing stream, whereas the ultrafiltration retentate comprises the biomass waste stream.

Any conventional ultrafiltration membrane can be used having a molecular weight cut-off (MWCO) range between about 1 and about 500 kDa, such as 10-250, 50-100, 200-500, 100-250, 1-100, 1-50, 10-25, 1-5 kDa, or any other suitable sub-range. The membrane material can be a ceramic or made of a synthetic or natural polymer, e.g. polysulfone, polyvinylidene fluoride, polyacrylonitrile, polypropylene, cellulose, cellulose acetate or polylactic acid. The ultrafiltration step can be applied in dead-end or cross-flow mode. Step I) of the method according to the invention may comprise more than one ultrafiltration step using membranes with different MWCO as defined above, e.g. applying two ultrafiltration separations, wherein the first membrane has a higher MWCO than that of the second membrane. This arrangement may provide a better separation efficacy of the higher molecular weight components of the broth. After this separation step, the permeate contains materials that have a molecular weight lower than the MWCO of the second membrane, including the neutral or sialylated human milk oligosaccharides of interest.

In one embodiment, the fermentation broth is ultrafiltered using a membrane having a MWCO of 5 to 30 kDa, such as 10-25, 15 or 20 kDa.

In a preferred embodiment, the yield of the desired neutral or sialylated HMO in the permeate after the ultrafiltration step performed in step I) is greater than 50%, greater than 60%, greater than 70%, greater than 80%, greater than 90%, greater than 91%, greater than 92%, greater than 93%, greater than 94%, greater than 95%, greater than 96%, greater than 97%, greater than 98%, or greater than 99%.

In another embodiment, the broth obtained from fermentation is subjected to centrifugation to separate the biomass from the neutral or sialylated HMO (HMO-containing stream) in step I) of the method according to the invention. In said embodiment, the supernatant represents the HMO-containing stream, while the remaining material, i.e. the “biomass waste stream” can be separated out. By centrifugation, a clear supernatant comprising the neutral or sialylated HMO can be obtained, which represents the HMO-containing stream.

The centrifuging can be lab scale or, advantageously over previous centrifuging methods, commercial scale (e.g. industrial scale, full production scale).

In some embodiments, a multi-step centrifugation can be used. For example, a series of 2, 3, 4, 5, 6, 7, 8, 9, or 10 centrifugation steps can be performed. In other embodiments, the centrifugation may be a single step. Centrifugation provides for a quick biomass-removal.

In certain embodiments, Sedicanter® centrifuge designed and manufactured by Flottweg can be used.

The particular type of centrifuge is not limiting, and many types of centrifuges can be used. The centrifuging can be a continuous process. In some embodiments, the centrifuging can have feed addition. For example, the centrifuging can have a continuous feed addition. In certain embodiments, the centrifuging can include a solid removal, such as a wet solid removal. The wet solid removal can be continuous in some implementations, and periodic in other implementations.

For example, a conical plate centrifuge (e.g. disk bowl centrifuge or disc stack separator) can be used. The conical plate centrifuge can be used to remove solids (usually impurities) from liquids, or to separate two liquid phases from each other by means of a high centrifugal force. The denser solids or liquids which are subjected to these forces move outwards towards the rotating bowl wall while the less dense fluids move towards the centre. The special plates (known as disc stacks) increase the surface settling area which speeds up the separation process. Different stack designs, arrangements and shapes are used for different processes depending on the type of feed present. The concentrated denser solid or liquid can then be removed continuously, manually, or intermittently, depending on the design of the conical plate centrifuge. This centrifuge is very suitable for clarifying liquids that have small proportion of suspended solids.

The centrifuge works by using the inclined plate setter principle. A set of parallel plates with a tilt angle θ with respect to horizontal plane is installed to reduce the distance of the particle settling. The reason for the tilted angle is to allow the settled solids on the plates to slide down by centrifugal force so they do not accumulate and clog the channel formed between adjacent plates.

This type of centrifuge can come in different designs, such as nozzle-type, manual-cleaning, self-cleaning, and hermetic. The particular centrifuge is not limiting.

Factors affecting the centrifuge include disk angle, effect of g-force, disk spacing, feed solids, cone angle for discharge, discharge frequency, and liquid discharge.

Alternatively, a solid bowl centrifuge (e.g. a decanter centrifuge) can be used. This is a type of centrifuge that uses the principle of sedimentation. A centrifuge is used to separate a mixture that consists of two substances with different densities by using the centrifugal force resulting from continuous rotation. It is normally used to separate solid-liquid, liquid-liquid, and solid-solid mixtures. One advantage of solid bowl centrifuges for industrial uses is the simplicity of installation compared to other types of centrifuge. There are three design types of solid bowl centrifuge, which are conical, cylindrical, and conical-cylindrical.

Solid bowl centrifuges can have a number of different designs, any of which can be used for the disclosed method. For example, conical solid bowl centrifuges, cylindrical solid bowl centrifuges, and conical-cylindrical bowl centrifuges can be used.

The centrifuging can be performed at a number of speeds and residence times. For example, the centrifuging can be performed with a relative centrifugal force (RCF) of 20000 g, 15000 g, 10000 g, or 5000 g. In some embodiments, the centrifuging can be performed with a relative centrifugal force (RCF) of less than 20000 g, 15000 g, 10000 g or 5000 g. In some embodiments, the centrifuging can be performed with a relative centrifugal force (RCF) of greater than 20000 g, 15000 g, 10000 g or 5000 g.

In some embodiments, the centrifuging can be characterized by working volume. In some embodiments, the working volume can be 1, 5, 10, 15, 20, 50, 100, 300, or 500 l. In some embodiments, the working volume can be less than 1, 5, 10, 15, 20, 50, 100, 300, or 500 l. In some embodiments, the working volume can be greater than 1, 5, 10, 15, 20, 50, 100, 300, or 500 l.

In some embodiments, the centrifuging can be characterized by feed flow rate. In some embodiments, the feed flow rate can be 100, 500, 1000, 1500, 2000, 5000, 10000, 20000, 40000, or 100000 l/hr. In some embodiments, the feed flow rate can be greater than 100, 500, 1000, 1500, 2000, 5000, 10000, 20000, 40000, or 100000 l/hr. In some embodiments, the feed flow rate can be less than 100, 500, 1000, 1500, 2000, 5000, 10000, 20000, 40000, or 100000 l/hr.

The amount of time spent centrifuging (e.g. residence time) can vary as well. For example, the residence time can be 0.1, 0.2, 0.5, 1, 2, 3, 4, 5, 6, 7, 8, 9, or 10 minutes. In some embodiments, the residence time can be greater than 0.1, 0.2, 0.5, 1, 2, 3, 4, 5, 6, 7, 8, 9, or 10 minutes. In some embodiments, the residence time can be less than 0.1, 0.2, 0.5, 1, 2, 3, 4, 5, 6, 7, 8, 9, or 10 minutes.

Any of the above supernatant properties can be produced through a single instance of centrifuging. Alternatively, it can be produced through multiple instances of centrifuging.

In view of the above, step I) of the method according to the invention can be performed via ultrafiltration as defined above or centrifugation, or via a combination of ultrafiltration and centrifugation. Preferably, method step I) is carried out by ultrafiltration as defined above to obtain the HMO-containing stream separate from the biomass waste stream.

Before the ultrafiltration and/or centrifugation step, the fermentation broth can be subjected to a pre-treatment step. Pre-treatment of the fermentation broth can include pH adjustment, and/or dilution, and/or heat treatment. In certain implementations, all three of pH adjustment, dilution, and heat treatment can be performed. In alternative embodiments, pH adjustment and dilution can be performed. In alternative embodiments, pH adjustment and heat treating can be performed. In alternative embodiments, heat treating and dilution can be performed.

Advantageously, a combination of a plurality of pre-treatment methods can provide an improved synergistic effect not found in individual pre-treatments.

In certain embodiments, one or more of the aforementioned pre-treatment steps can occur during the biomass removal in step I) by centrifuging and/or ultrafiltration as defined above. For example, between steps in a multi-step centrifuging, or the centrifuging vessel may be able to heat the fermentation broth during centrifuging.

Advantageously, the pre-treatment can increase the settling velocity of the solid particles (biomass) in the fermentation broth by a factor of 100 to 20000, making the biomass separation by centrifugation much more efficient and thus applicable in industrial scale. In addition to settling velocity, at least three other parameters are substantially improved due to pre-treatment that are, improved neutral or sialylated HMO yield in the HMO-containing stream, reduced protein and DNA content in the supernatant, and further residual suspended solid content can be substantially reduced.

Purifying the HMO-containing stream in step II) In step II) of the method according to the invention, the HMO-containing stream is purified by nanofiltration then with an acidic cation exchange resin treatment, or purified with an acidic cation exchange resin treatment then by nanofiltration.

Nanofiltration (NF) can be used to remove low molecular weight molecules smaller than the desired neutral or sialylated HMOs, such as mono- and disaccharides, short peptides, small organic acids, water, and salts.

The product stream, i.e. the HMO-containing steam, is the NF retentate. The nanofiltration membrane thus has a MWCO that ensures the retention of the neutral or sialylated of interest, i.e. the MWCO of the nanofiltration membrane is adjusted accordingly.

Typically, the pore size of the nanofiltration membrane is from 0.5 nm to 2 nm and/or from 150 dalton (Da) molecular weight cut-off (MWCO) to 3000 Da MWCO.

In an embodiment, the membranes are in the range of 150-300 Da MWCO, which are defined as “tight” NF membranes.

In a preferred embodiment, the membranes are above 300 Da MWCO, and preferably not higher than 3000 Da MWCO. In said embodiment, the membranes are considered “loose” NF membranes.

In another preferred embodiment, the “loose” nanofiltration membrane has a molecular weight cut-off (MWCO) of 500-3000 Da and the active (top) layer of the membrane is preferably composed of polyamide, more preferably piperazine-based polyamide. Thereby, the retention of tri- or higher oligosaccharides is ensured and at least part of the disaccharides is allowed to pass the membrane. In this embodiment, the applied nanofiltration membrane shall be tight for tri- and higher oligosaccharides for them to be efficiently retained. At the same time, the membrane shall be relatively loose for MgSO₄, that its rejection is about 50-90%, in order that disaccharides can pass the membrane. This way, it is possible to separate e.g. lactose, which is a precursor in making human milk oligosaccharides e.g. by fermentation, from the neutral or sialylated human milk oligosaccharides product with a good efficacy, and additionally a substantial part of divalent ions also passes to the permeate. In some embodiments, the MgSO₄ rejection factor is 60-90%, 70-90%, 50-80%, 50-70%, 60-70% or 70-80%. Preferably, the MgSO₄ rejection factor on said membrane is 80-90%. Preferably, the membrane has a rejection factor for NaCl that is lower than that for MgSO₄. In one embodiment, the rejection factor for NaCl is not more than 50%. In another embodiment, the rejection factor for NaCl is not more than 40%. In another embodiment, the rejection factor for NaCl is not more than 30%. In this latter embodiment, a substantial reduction of all monovalent salts in the retentate is also achievable. In said embodiment, the membrane is a thin-film composite (TFC) membrane. An example of a suitable piperazine-based polyamide TFC membrane is TriSep® UA60. Other examples of suitable NF membranes include Synder NFG (600-800 Da), Synder NDX (500-700 Da), and TriSep® XN-45 (500 Da).

Preferably, the yield of the desired neutral or sialylated HMO in the retentate after the nanofiltration step is greater than 50%, greater than 60%, greater than 70%, greater than 80%, greater than 90%, greater than 91%, greater than 92%, greater than 93%, greater than 94%, greater than 95%, greater than 96%, greater than 97%, greater than 98%, or greater than 99%.

In a preferred embodiment, the nanofiltration step further comprises a diafiltration step, that is the nanofiltration is conducted in diafiltration mode. Preferably, the diafiltration follows the aforementioned (conventionally conducted) nanofiltration step.

Diafiltration is a process that involves the addition of purified water to a solution during membrane filtration process in order to remove membrane permeable components more efficiently. Thus, diafiltration can be used to separate components on the basis of their properties, in particular molecular size, charge or polarity by using appropriate membranes, wherein one or more species are efficiently retained and other species are membrane permeable.

In a preferred embodiment, diafiltration and nanofiltration can be combined within one step (referred to as nanofiltration/diafiltration or NF/DF) in which diafiltration is executed while using a nanofiltration membrane that is effective for the separation of low molecular weight compounds and/or salts from the neutral or sialylated HMOs. Diafiltration with “loose” NF membrane as defined above, is particularly efficient for both mono- and divalent salts removal and disaccharides removal from neutral or sialylated HMOs.

In a preferred embodiment, using a “loose” nanofiltration membrane as disclosed above, the DF step or the NF/DF step is performed so that the pH is set below 5.0, preferably, below 4.5, advantageously below 4.0, but preferably not less than 3.0. The condition ensures the retention of the neutral or sialylated HMO to be purified and allowing the mono- and divalent salts to pass and accumulate in the permeate, and also allowing at least a part of lactose to pass and accumulate in the permeate. Therefore salts of monovalent cations such as sodium salts (that is sodium ion together with the co-anion(s)) are effectively removed, giving rise to a low-salt or a practically salt-free purified solution containing a neutral or sialylated HMO in the retentate.

The method according to the invention comprises further purification of the HMO-containing stream with an acidic cation exchange resin in step II).

In the cation exchanger treatment step, positively charged materials can be efficiently removed from the HMO-containing stream, either before or after nanofiltration, as they bind to the resin, while the neutral or sialylated HMOs will not be retained by the acidic cation exchange resin.

Thereby, also the amounts of salts and/or colorizing agents and/or proteins can be further reduced.

In a preferred embodiment, the stationary phase (resin) comprises sulfonate groups that are negatively charged in aqueous solution and that tightly bind cationic compounds.

In a preferred embodiment, the acidic cation exchange resin is a strongly acidic cation exchange resin, preferably a polystyrene-divinylbenzene cation exchange resin.

In a further preferred embodiment, the acidic cation exchange resin is in H⁺-form.

The binding capacity of an acidic cation exchange resin is generally from 1.2 to 2.2 eq/l.

When using a cationic ion exchange resin, its degree of crosslinking can be chosen depending on the operating conditions of the ion exchange column. A highly crosslinked resin offers the advantage of durability and a high degree of mechanical integrity, however, suffers from a decreased porosity and a drop off in mass-transfer. A low-crosslinked resin is more fragile and tends to swell by absorption of mobile phase. The particle size of the ion exchange resin is selected to allow an efficient flow of the eluent, while the charged materials are still effectively removed. A suitable flow rate may also be obtained by applying a negative pressure to the eluting end of the column or a positive pressure to the loading end of the column, and collecting the eluent. A combination of both positive and negative pressure may also be used. The cationic ion exchange resin treatment can be carried out in a conventional manner, e.g. batch-wise or continuously.

Non-limiting examples of a suitable acidic cation exchange resin can be e.g. Amberlite IR100, Amberlite IR120, Amberlite FPC22, Dowex 50WX, Finex CS16GC, Finex CS13GC, Finex CS12GC, Finex CS11GC, Lewatit S, Diaion SK, Diaion UBK, Amberjet 1000, Amberjet 1200.

Preferably, the cation exchange resin treatment step is performed after the nanofiltration step. However, said cation exchange resin treatment step can also be conducted after a further optional step making use of active carbon as further described below.

In a preferred embodiment, step II) results in a purified solution containing the neutral or sialylated HMO at a purity of ≥80%, preferably ≥85%, more preferably ≥90%.

In a preferred embodiment, step II) results in a purified solution that is free of proteins and/or recombinant genetic material.

In an even preferred embodiment, a second nanofiltration/diafiltration step is carried out in step II) of the method according to the invention. In said second nanofiltration step, the nanofiltration membrane is a “loose” NF membrane, see above. The second optional NF/DF step is performed after the first nanofiltration step, but is preferably performed before step III) of the method according to the invention. The second nanofiltration is preferably performed in diafiltration mode. This second NF/DF step is performed so that the pH is set below 5.0, preferably below 4.5, advantageously below 4.0, but preferably not less than 3.0.

In a particularly preferred embodiment, step II) comprises a first NF or NF/DF purification of the HMO-containing stream obtained in step I) then strong cation exchange resin treatment (H⁺-form) of the retentate from the first NF or NF/DF step, setting the pH of the resin eluate with NaOH-solution below 5.0, preferably below 4.5, advantageously below 4.0, but preferably not less than 3.0, then performing a second NF/DF step. As cation exchanger removes effectively cations, only sodium ion is reintroduced after neutralization. The present inventors surprisingly found that the rejection of (inorganic) sodium salts even with divalent counter-anions at pH below 5.0, preferably below 4.5 is low when a “loose” NF membrane as disclosed above was used in NF/DF. In this regard, the sodium ions bring the anions to the permeate, that is the sodium salts are easily permeable, making it possible that an aqueous solution of the neutral or sialylated HMO is collected in the retentate that is practically salt-free, but at least has a very low salt content. Therefore, the use of basic anion exchangers is avoidable in the purification of neutral or sialylated HMOs.

### Concentrating the Purified HMO-Containing Stream in Step III) of the Method According to the Invention

A concentration step is used to economically remove significant quantities of liquid, mostly water, from the neutral or sialylated HMO-containing stream using e.g. evaporation, nanofiltration, or reverse-osmosis filtration. Evaporation processes can include, e.g. falling film evaporation, climbing film evaporation and rotary evaporation. The evaporation can also be conducted under vacuum. The incoming solids concentration to the process is preferably approximately 5 to 30 wt. %. The exit solids concentration from such a process is typically greater than 30 wt. %., preferably greater than 50 wt. %. More preferably, the solids concentration exiting the dewatering operation is 60 to 80 wt. %. The solids portion of the recovered material is preferably greater than 80 wt. % of neutral or sialylated HMO.

In an embodiment, the purified neutral or sialylated HMO-containing stream is concentrated to a concentration of ≥100 g/l of neutral or sialylated HMO, preferably of ≥200 g/l, more preferably of ≥300 g/l.

When the purified neutral or sialylated HMO-containing stream is concentrated by evaporation, the evaporation is preferably carried out at a temperature of from about 20 to about 80° C. In some embodiments, the evaporation is carried out at a temperature of from 25 to 75° C. In some embodiments, the evaporation is carried out at a temperature of from 30 to 70° C. In some embodiments, the evaporation is carried out at a temperature of from 30 to 65° C. Preferably, the evaporation is carried out under vacuum.

When the purified neutral or sialylated HMO-containing stream is concentrated by membrane filtration, any membrane, typically nanofiltration membrane, is suitable that sufficiently rejects the neutral or sialylated HMO. Concentration by membrane filtration usually provides an HMO-solution of around 30-35 wt %. This concentration may be suitable for conducting the subsequent drying-solidification step, e.g. freeze-drying. However, other drying methods may require more concentrated solutions, e.g. spray-drying or crystallization. In this case, concentration by evaporation, preferably under vacuum, is the preferred embodiment. Alternatively, the neutral or sialylated HMO-containing stream obtained in the previous step is concentrated to around 30-35 wt % using a nanofiltration membrane, and the solution is further concentrated by evaporation.

In one embodiment of the concentration by membrane filtration, the membrane of choice is a “tight” NF with 150-300 Da MWCO.

In other embodiment of the concentration by membrane filtration, the membrane of choice is a nanofiltration membrane that has a molecular weight cut-off (MWCO) of 500-3500 Da and an active (top) layer of polyamide (“loose” NF membrane); and the concentration step is performed so that the pH is set below 5.0, preferably below 4.5, advantageously below 4.0, but preferably not less than 3.0. In this latter embodiment, a substantial reduction of all monovalent salts in the retentate is also achievable. In said embodiment, the membrane is preferably a thin-film composite (TFC) membrane which is a piperazine-based polyamide membrane, more preferably its MgSO₄ rejection is about 50-90%, even more preferably its NaCl rejection is not more than 50%. An example of such a membrane is TriSep® UA60. Under this condition, remaining salts are also effectively removed, giving rise to a low-salt or a practically salt-free purified neutral or sialylated HMO-concentrate. In this embodiment, after completing the concentration step, the pH of the neutral or sialylated HMO-concentrate is advantageously set between 4-6 before performing the next step (e.g. evaporation, drying-solidification, sterile filtration).

The concentration step may be optional when step IV) is freeze-drying.

### Drying the Purified HMO-Containing Stream to Obtain a Solidified Neutral or Sialylated HMO in Step IV)

Preferably after the separation/purification/concentration steps according to method steps I)-III) and any of the undermentioned optional method steps, respectively, the neutral or sialylated HMO of interest is provided in its solid form via a drying step (step IV)).

In a preferred embodiment, drying step IV) comprises spray-drying of the neutral or sialylated HMO-containing stream, preferably consists of spray-drying of the neutral or sialylated HMO-containing stream.

Preferably, spray-drying leads to solidified neutral or sialylated HMO having an amorphous structure, i.e. an amorphous powder is obtained.

In an embodiment, spray-drying is performed at a concentration of the neutral or sialylated HMO of 20-60% (w/v), preferably 30-50% (w/v), more preferably 35-45% (w/v), and an inlet temperature of 110-150° C., preferably 120-140° C., more preferably 125-135° C. and/or an outlet temperature of 60-80° C., preferably 65-70° C.

In some embodiment, the neutral or sialylated HMO-containing stream fed into the spray-dryer has a Brix value of from about 8 to about 75% Brix. In some embodiments, the Brix value is from about 30 to about 65% Brix. In some embodiments, the Brix value is from about 50 to about 60% Brix. In some embodiments, the feed into the spray-dryer is at a temperature of from about 2 to about 70° C. immediately before being dispersed into droplets in the spray-dryer. In some embodiments, the feed into the spray-dryer is at a temperature of from about 30 to about 60° C. immediately before being dispersed into droplets in the spray-dryer. In some embodiments, the feed into the spray-dryer is at a temperature of from about 2 to about 30° C. immediately before being dispersed into droplets in the spray-dryer. In some embodiments, the spray-drying uses air having an air inlet temperature of from 120 to 280° C. In some embodiments, the air inlet temperature is from 120 to 210° C. In some embodiments, the air inlet temperature is from about 130 to about 190° C. In some embodiments, the air inlet temperature is from about 135 to about 160° C. In some embodiments, the spray-drying uses air having an air outlet temperature of from about 80 to about 110° C. In some embodiments, the air outlet temperature is from about 100 to about 110° C. In some embodiments, the spray-drying is carried out at a temperature of from about 20 to about 90° C. In some embodiments, the spray-dryer is a co-current spray-dryer.

In some embodiments, the spray-dryer is attached to an external fluid bed. In some embodiments, the spray-dryer comprises a rotary disk, a high-pressure nozzle, or a two-fluid nozzle. In some embodiments, the spray-dryer comprises an atomizer wheel. In some embodiments, spray-drying is the final purification step for the desired neutral or sialylated HMO.

Alternatively, the drying-solidification step comprises an indirect drying method. For the purposes of this specification, indirect dryers include those devices that do not utilize direct contact of the material to be dried with a heated process gas for drying, but instead rely on heat transfer either through walls of the dryer, e.g. through the shell walls in the case of a drum dryer, or alternately through the walls of hollow paddles of a paddle dryer, as they rotate through the solids while the heat transfer medium circulates in the hollow interior of the paddles. Other examples of indirect dryers include contact dryers and vacuum drum dryers.

Alternatively, the drying-solidification step comprises freeze-drying.

Alternatively, the drying-solidification step comprises crystallization (provided that the HMO is obtainable in crystalline form).

### Optional Steps

In a preferred embodiment, the method according to the invention further comprises purification of by an active carbon treatment.

The treatment with active carbon represents a decolorization step (removing colorizing components) and/or a chromatographic step on a neutral solid phase, preferably reversed-phase chromatography to remove hydrophobic contaminants. Preferably, active carbon, such as Norit CA1 activated carbon can be used.

The active carbon treatment may serve to remove colorizing agents and may further reduce the amounts of water-soluble contaminants, such as salts. Moreover, the active carbon treatment may serve to remove proteins, DNAs, RNAs, or endotoxin that may be present in the HMO-containing stream.

Hence, the active carbon treatment leads to a reduction of colorizing agents and/or salts and/or proteins and/or DNAs and/or RNAs and/or endotoxin in the HMO-containing stream.

Under certain conditions, the neutral or sialylated human milk oligosaccharides do not, or at least not substantially, adsorb to the carbon particles and elution with water gives rise to an aqueous solution of the neutral or sialylated human milk oligosaccharides without a significant loss in their amounts, while colorizing agents, proteins, DNAs, RNAs, endotoxin, etc. remain adsorbed. It is merely a matter of routine skills to determine the conditions under which the neutral or sialylated human milk oligosaccharides would bind to the carbon from its aqueous solution.

Hence, the optional active charcoal treatment step is performed so that the neutral or sialylated HMO is not or at least not substantially adsorbed by the active carbon. Under “not substantially adsorbed”, it is understood that less than 10%, preferably less than 5%, and more preferably less than 1% of the neutral or sialylated HMO is adsorbed by the active carbon. The amount of active carbon used in this aspect is <100% by weight relative to the neutral or sialylated HMO being present in the HMO-containing stream, preferably <10%. This can allow most of the neutral or sialylated HMO to pass while residual biomolecules, coloured compounds, and other hydrophobic molecules, are retaining by the active carbon. In an embodiment, the amount of the applied active carbon is around 2-6 wt. %. This is economical, because all the benefits disclosed above can be conveniently achieved with a very low amount of carbon. In other embodiment, the active carbon is added in an amount in the range of 0.25 wt. % to 3 wt. %, preferably in the range of 0.5 wt. % to 2.5 w.t %, and more preferably in the range of 0.75 wt. % to 2.2 wt. %, and even more preferably in the range of 1.0 wt. % to 2.0 wt. %, wherein the percentage values are based on the total weight of the HMO-containing stream that is subjected to the active carbon treatment step. This rather small amount of active carbon allows for significant reduction of active carbon consumption as well as for a significant reduction of product losses (neutral or sialylated HMO).

In one aspect, the active carbon treatment can be conducted by adding carbon powder to the HMO-containing steam under stirring and filtering off the carbon.

In other aspect, for higher scale purification, the aqueous solution containing the neutral or sialylated human milk oligosaccharide (HMO-containing stream) is preferably loaded to a column packed with carbon, which may be a granulated carbon or may optionally be mixed with inert filter aid, then the column is washed with the required eluent. The fractions containing the neutral or sialylated human milk oligosaccharide are collected.

In one embodiment, the active carbon used is granulated. This ensures a convenient flow-rate without applying high pressure.

In one embodiment, the active carbon treatment, preferably comprising active carbon chromatography is conducted at elevated temperature. At elevated temperature, the binding of colorizing agents, residual proteins, etc. to the carbon particles takes place in a shorter contact time, therefore the flow-rate can be conveniently raised. Moreover, the active carbon treatment conducted at elevated temperature substantially reduces the total number of viable microorganisms (total microbial count) in the HMO-containing stream. The elevated temperature may be at least 30-35° C., such as at least 40° C., at least 50° C., around 40-50° C., or around 60° C.

In one embodiment, the active carbon is added as a powder having a particle size distribution with a diameter d50 in the range of 2 μm to 25 μm, preferably in the range of 3 μm to 20 μm, and more preferably in the range of 3 μm to 7 μm, and even more preferably in the range of 5 μm to 7 μm. The d50 value is determined with standard procedures.

In one embodiment, the pH of the HMO-containing stream is adjusted before the active carbon treatment is carried out to improve the reduction of colorizing agents and/or proteins during step II) of the method according to the invention. Preferably, the pH is adjusted to 5.5, more preferably to 5.0 and even more preferably to 4.5 by the addition of a suitable acid.

The optional active carbon treatment may follow the cation exchange resin treatment in step II) and is preferably conducted before step III) of the method according to the invention. In case an optional second nanofiltration or nanofiltration/diafiltration step is performed as described above, the optional active carbon treatment can be performed before or after said optional second nanofiltration or nanofiltration/diafiltration step, but preferably before.

In another embodiment, the method according to the invention further comprises a step, wherein the HMO-containing solution, preferably after concentration according to step III), is sterile filtered and/or subjected to endotoxin removal, preferably by filtration of the purified solution through a 3 kDa filter. Said optional step is preferably conducted after step II) and any of the aforementioned optional purification steps and before the drying step according to step IV).

According to one embodiment, both the active charcoal treatment and the sterile filtration step, disclosed above, are part of the method of the invention.

### Particular Embodiments of the Invention

In preferred embodiments, the method according to the present invention does not include a basic anion exchange resin treatment step.

In preferred embodiments, the method according to the present invention does not include an electrodialysis step.

In preferred embodiments, the method according to the present invention does not include an electrodialysis step and a basic anion exchange resin treatment step.

In a preferred embodiment, the method according to the invention comprises or consists of the following steps (in consecutive order):
i. separating the fermentation broth to form a separated HMO-containing stream and a biomass waste stream by ultrafiltration;
ii. purifying the separated HMO-containing stream by combined nanofiltration and diafiltration, wherein the nanofiltration membrane is preferably in the range of 500-3000 Da MWCO;
iii. purifying the nanofiltration retentate by a strongly acidic cation exchange resin in H⁺-form;
iv. purifying the resin eluate by a second nanofiltration step, preferably combined with diafiltration, wherein the nanofiltration membrane has a molecular weight cut-off (MWCO) of 500-3000 Da, the active (top) layer of the membrane is composed of polyamide, more preferably piperazine-based polyamide, the membrane has a MgSO₄ rejection factor of about 50-90% and preferably a NaCl rejection factor of not more than 50%, and the nanofiltration step is performed so that the pH is set below 5.0, preferably below 4.5, advantageously below 4.0, but preferably not less than 3.0;
v. concentrating the nanofiltration retentate by evaporation or reverse osmosis; and
vi. spray-drying the concentrate to obtain a solidified neutral or sialylated HMO, optionally with active charcoal treatment, preferably after step ii), iii), iv) or v), more preferably between steps iii) and iv).

Step iii) comprises the addition of NaOH-solution to the acidic resin eluate so that the pH is set to 3-5.

In another preferred embodiment, the method according to the invention comprises or consists of the following steps (in consecutive order):
i. separating the fermentation broth to form a separated HMO-containing stream and a biomass waste stream by ultrafiltration;
ii. purifying the separated HMO-containing stream by combined nanofiltration and diafiltration, wherein the nanofiltration membrane is preferably in the range of 500-3000 Da MWCO;
iii. purifying the nanofiltration retentate by a strongly acidic cation exchange resin in H⁺-form;
iv. purifying the resin eluate by a second nanofiltration step, preferably combined with diafiltration, wherein the nanofiltration membrane has a molecular weight cut-off (MWCO) of 500-3000 Da, the active (top) layer of the membrane is composed of polyamide, more preferably piperazine-based polyamide, the membrane has a MgSO₄ rejection factor of about 50-90% and preferably a NaCl rejection factor of not more than 50%, and the nanofiltration step is performed so that the pH is set below 5.0, preferably below 4.5, advantageously below 4.0, but preferably not less than 3.0;
v. optionally concentrating the nanofiltration retentate by evaporation or reverse osmosis; and
vi. freeze-drying the nanofiltration retentate or the concentrate to obtain a solidified neutral or sialylated HMO, optionally with active charcoal treatment, preferably after step ii), iii), iv) or v), more preferably between steps iii) and iv).

Step iii) comprises the addition of NaOH-solution to the acidic resin eluate so that the pH is set to 3-5.

### 3. Neutral or Sialylated Human Milk Oligosaccharide Produced by the Method According to the Invention

In another aspect, the invention relates to a neutral or sialylated human milk oligosaccharide obtained by the method according to the invention.

The neutral or sialylated HMO recovered and purified according to the method described in this specification can be amorphous or crystalline, preferably amorphous.

In a preferred embodiment, the purity of the neutral or sialylated HMO on a dry basis is greater than 80 wt. % for a single neutral or sialylated HMO based on dry matter; or for mixtures of HMOs, greater than 70% purity based on dry matter, for the combination. More preferably, the purity of a single neutral or sialylated HMO is greater than 90 wt. %.

In a preferred embodiment, the neutral or sialylated HMO has at least one of the following characteristics (by weight): <2% lactulose, <3% fucose, <1% galactose, or <3% glucose.

In an embodiment, the neutral or sialylated HMO has a fines fraction (less than or equal to 10 μm), of less than 10%, preferably less than 5%, more preferably less than 1%, most preferably less than 0.1%. The neutral or sialylated HMO also preferably has an average particle size (d50), of greater than 100 μm, more preferably greater than 150 μm, even more preferably greater than 200 μm.

The neutral or sialylated HMO produced by the method according to the invention, demonstrates good flowability. Preferably, the HMO has a Carr index of less than 30, where the Carr index (C) is determined by the formula C=100(1−ρB/ρT), where ρB is the freely settled bulk density of the powder, and ρ T is the tapped bulk density of the powder after “tapping down”. For free-flowing solids, the values bulk and tapped density would be similar, so the value is small. For poorer flowing solids, the differences between these values would be larger, so that the Carr index would be larger.

In a preferred embodiment, the neutral or sialylated HMO has a water content of less than 15 wt. %, less than 10 wt. %, less than 9 wt. %, less than 8 wt. %, less than 7 wt. %, or less than 6 wt. %. In order to optimize product recovery, preferably, the neutral or sialylated HMO has a pH greater than 3.0 in at least 5% solution. Typically, this is achieved by adjusting the pH of the HMO-containing stream to greater than 3.0 prior to the drying step. Preferably, the neutral or sialylated HMO has a pH of from 4 to 7, more preferably from 4.5 to 5.5.

Preferably, the HMO is a neutral HMO. In an embodiment, the neutral HMO is preferably selected from the group consisting of 2′-fucosyllactose, 3-fucosyllactose, 2′,3-difucosyllactose, lacto-N-triose II, lacto-N-tetraose, lacto-N-neotetraose, lacto-N-fucopentaose I, lacto-N-fucopentaose II, lacto-N-fucopentaose III, lacto-N-fucopentaose V, lacto-N-neofucopentaose V (alternative name: lacto-N-fucopentaose VI), lacto-N-difucohexaose I, lacto-N-difucohexaose II, lacto-N-difucohexaose III, 6′-galactosyllactose, 3′-galactosyllactose, lacto-N-hexaose, lacto-N-neohexaose, and any mixture thereof. More preferably, the HMO is 2′-fucosyllactose, 3-fucosyllactose, 2′,3-difucosyllactose, lacto-N-triose II, lacto-N-tetraose, lacto-N-neotetraose or a lacto-N-fucopentaose, more preferably 2′-fucosyllactose, LNT, LNnT or a lacto-N-fucopentaose.

In an embodiment, the sialylated HMO is selected from the group consisting of 3′-sialyllactose (3′-SL) and 6′-sialyllactose (6′-SL).

In an embodiment, the neutral or sialylated HMO obtained by the method according to the invention, is incorporated into a food product (e.g. human or pet food), dietary supplement or medicine product.

In some embodiments, the neutral or sialylated HMO is incorporated into a human baby food (e.g. infant formula). Infant formula is generally a manufactured food for feeding to infants as a complete or partial substitute for human breast milk. In some embodiments, infant formula is sold as a powder and prepared for bottle- or cup-feeding to an infant by mixing with water. The composition of infant formula is typically designed to roughly mimic human breast milk. In some embodiments, a neutral or sialylated HMO purified by a method in this specification is included in infant formula to provide nutritional benefits similar to those provided by one or more neutral or sialylated HMOs in human breast milk. In some embodiments, the neutral or sialylated HMO is mixed with one or more ingredients of the infant formula. Examples of infant formula ingredients include skimmed milk, carbohydrate sources (e.g. lactose), protein sources (e.g. whey protein concentrate and casein), fat sources (e.g. vegetable oils—such as palm, high oleic safflower oil, rapeseed, coconut and/or sunflower oil; and fish oils), vitamins (such as vitamins A, B, B2, C and D), minerals (such as potassium citrate, calcium citrate, magnesium chloride, sodium chloride, sodium citrate and calcium phosphate).

Hence, another aspect of the invention relates to a neutral or sialylated human milk oligosaccharide obtained by the method according to the invention for use in medicine.

Hence, another aspect of the invention relates to the use of a neutral or sialylated human milk oligosaccharide obtained by the method according to the invention for food and/or feed applications.

Hence, another aspect of the invention relates to a food or cosmetic product comprising the neutral or sialylated human milk oligosaccharide obtained by the method according to the invention.

### EXAMPLES

### Example 1

General: Carbohydrate and impurity content were quantified by calibrated HPLC and/or HPAEC. Soluble proteins were quantified by Bradford assay. Colour was quantified by UV-absorption measurement at 400 nm in a cuvette with 1 cm path. The colour index CI_400 is calculated according to the following formula: CI_400=1000*Abs_400/Brix.

Fermentation: 2′-FL was produced by microbial fermentation using a genetically modified E. coli strain comprising a recombinant gene encoding an α-1,2-fucosyltransferase. The fermentation was performed by culturing the strain in the presence of exogenously added lactose and a suitable carbon source, thereby producing 2′-FL which was accompanied with DFL and unreacted lactose as major carbohydrate impurities in the fermentation broth.

### Purification:

1. UF/DF: The obtained broth was acidified to pH=3.8 with sulphuric acid followed by ultrafiltration-diafiltration through 15 kDa ceramic membrane elements at T=+60° C. in industrial continuous UF system with the DF water flow of approximately 2-times the feed flow and UF retentate flow of ca. half of the feed flow.
2. NF/DF: The UF permeate stream containing the product (Brix ca. 5) was immediately processed by loose nanofiltration with diafiltration in industrial continuous NF system equipped with Trisep UA60 membrane (piperazine-amide, MWCO 1000-3500 Da) elements (trans membrane pressure, TMP=30 bar, T=15° C.) and with a DF water flow approximately twice as the feed (UF permeate) flow.
3. Strong cation exchange resin treatment: A sample of the obtained NF retentate (4.7 kg, Brix 23.8, conductivity: 2.20 mS/cm, pH=3.9, CI_400: 134) was passed through a column packed with 800 ml of Dowex-88 resin in H⁺-form (capacity 1.8 eq/l) at 2 bed volumes per hour flow rate followed by elution with water (730 ml). 400 ml fractions were collected. Each fraction was titrated with 1M NaOH to pH=4.5 and analysed for sugar, colour and protein content. The pH-adjusted fractions #2-13 were combined to give 5.4 kg of yellow solution (Brix 20.2, conductivity: 3.48 mS/cm, pH=4.55, CI_400: 67.3).
4. Active charcoal decolorization: Part of the obtained solution (3.5 kg) was passed through granulated active charcoal CPG LF (75 g, 150 ml) packed in a column (ID=16 mm) at +60° C. and at 2 bed volumes per hour flow rate followed by water elution (300 ml). 150 ml fractions were collected. Fractions #2-24 were combined to give 3.5 kg of nearly colourless solution (Brix 19.7, conductivity 3.42 mS/cm, pH=5.19, CI_400: 1.23).
5. NF/DF: The obtained solution (3.4 kg) was subjected to constant volume diafiltration in MMS SW18 membrane filtration system equipped with 1812-size spiral wound Trisep UA60 membrane under the following conditions: cross-flow=400 l/h, TMP=30 bar, T=30-35° C. and DF water flow 4.0 l/h. First, the DF was performed at a pH of around 5 (10 1), then at 3.8 (extra 10 1). Finally, the retentate was further concentrated at TMP=39 bar followed by a pH adjustment to 4.5 with 4% NaOH solution and pumped out from the system to give 1.8 kg of a final solution (Brix 30.6, conductivity: 0.216 mS/cm, pH=4.49, CI_400: 1.35).
6. Microfiltration and freeze-drying: The above obtained NF retentate was passed through a PES 0.2 μm micro-filter to give 1.6 kg of solution (Brix 30.6) and freeze-dried to give 492 g of a white powder.

### Example 2: Comparison of MgSO₄ and Na₂SO₄ Rejection

2.01 of 0.2% MgSO₄ solution were loaded into a MMS SW18 system equipped with 1812-size spiral wound Trisep UA60 element (piperazine-amide, MWCO 1000-3500 Da, membrane area 0.23 m²). The system was run at 400 l/h cross-flow with permeate circulating back to the feed tank. It was equilibrated for at least 5 min or until constant conductivity in the permeate under each condition. The pH was adjusted by adding a small amount of 25% H₂SO₄ solution. The conductivity of the permeate and the retentate are disclosed in the table below.

MgSO₄ rejection vs pH

| | | 25% | | | | | | |
|---|---|---|---|---|---|---|---|---|
| | | H₂SO₄ | | | | Retentate | Permeate | |
| TMP | T | added | pH | Flow | Flux | conductivity | conductivity | Rejection |
| (bar) | (° C.) | (μl) | (retentate) | (l/h) | (l/m²h) | (mS/cm) | (mS/cm) | factor |
| 10 | 24.7 | 0 | 6.06 | 21.1 | 91.6 | 2.100 | 0.785 | 62.61% |
| 10 | 25.2 | 20 | 4.91 | 20.9 | 90.8 | 2.100 | 0.587 | 72.05% |
| 10 | 24.9 | 40 | 4.35 | 20.9 | 90.8 | 2.130 | 0.624 | 70.70% |
| 10 | 25.1 | 80 | 3.93 | 20.5 | 89.2 | 2.170 | 0.520 | 76.04% |
| 10 | 25.1 | 160 | 3.56 | 20.2 | 87.7 | 2.260 | 0.538 | 76.19% |

The same experiment was performed with 0.2% Na₂SO₄ solution.

Na₂SO₄ rejection vs pH

| | | 25% | | | | | | |
|---|---|---|---|---|---|---|---|---|
| | | H₂SO₄ | | | | Retentate | Permeate | |
| TMP | T | added | pH | Flow | Flux | conductivity | conductivity | Rejection |
| (bar) | (° C.) | (μl) | (retentate) | (l/h) | (l/m²h) | (mS/cm) | (mS/cm) | factor |
| 10 | 22.0 | 0 | 5.70 | 16.7 | 72.6 | 2.840 | 0.0755 | 97.34% |
| 10 | 22.2 | 10 | 5.24 | 15.6 | 67.8 | 2.840 | 0.1706 | 93.99% |
| 10 | 22.0 | 30 | 4.39 | n/a | n/a | 2.840 | 0.824 | 70.99% |
| 10 | 22.2 | 70 | 3.91 | 13.6 | 59.1 | 2.850 | 1.706 | 40.14% |
| 10 | 22.2 | 110 | 3.71 | 18.0 | 78.3 | 2.880 | 1.939 | 32.67% |
| 10 | 22.0 | 190 | 3.48 | 18.4 | 80.0 | 2.970 | 2.090 | 28.67% |
| 10 | 22.0 | 350 | 3.21 | 18.4 | 80.0 | 3.060 | 2.230 | 27.12% |
| 10 | 22.0 | 670 | 2.94 | 18.4 | 80.0 | 3.280 | 2.500 | 23.78% |
| 10 | 22.0 | 1310 | 2.65 | 18.1 | 78.7 | 3.730 | 2.870 | 23.06% |

It was demonstrated that the sodium salt rejection with divalent counter-ion such as sulphate is strongly pH dependent in case of NF with polyamide membrane. Because a substantial amount of sodium salt is present in the collected fractions after the strong cation exchange resin treatment due to neutralization with NaOH solution (see step #3 in Example 1), these salts can be effectively removed in a second NF/DF (see step #5 in Example 1) when the DF is conducted at a pH of less than 4.5, advantageously less than 4.0, resulting in a practically salt-free solution (as assessed from conductivity). In this regard, no basic anionic resins are necessary to use to obtain a salt-free solution.

### Example 3—Determination of a Substance Rejection Factor on a Membrane

The NaCl and MgSO₄ rejection on a membrane is determined as follows: in a membrane filtration system, a NaCl (0.1%) or a MgSO₄ (0.2%) solution is circulated across the selected membrane sheet (for Tami: tubular module) while the permeate stream is circulated back into the feed tank. The system is equilibrated at 10 bars and 25° C. for 10 minutes before taking samples from the permeate and retentate. The rejection factor is calculated from the measured conductivity of the samples: (1−κₚ/κᵣ) 100, wherein κₚ is the conductivity of NaCl or MgSO₄ in the permeate and κᵣ is the conductivity of NaCl or MgSO₄ in the retentate.

| | | | NaCl rej. factor | | MgSO₄ rej. factor | |
|---|---|---|---|---|---|---|
| | | | supplier | lab. | supplier | lab. |
| membrane | active layer | MWCO | spec. | measurement | spec. | measurement |
| Trisep | piperazine- | 1000- | — | 10% | 80% | 81-89% |
| UA60 | PA | 3500 | | | | |
| Tami | ceramic | 1000 | — | — | — | 0% |

A carbohydrate rejection factor is determined in a similar way with the difference that the rejection factor is calculated from the concentration of the samples (determined by HPLC): (1−Cₚ/Cᵣ) 100, wherein Cₚ is the concentration of the carbohydrate in the permeate and Cᵣ is the concentration of the carbohydrate in the retentate.

### Example 4

Fermentation: LNT-containing broth was generated by fermentation as described above using a genetically modified E. coli strain of LacZ⁻, LacY⁺ phenotype, wherein said strain comprised a recombinant gene encoding β-1,3-N-acetyl-glucosaminyl transferase which is able to transfer the GlcNAc of UDP-GlcNAc to the internalized lactose, a recombinant gene encoding a β-1,3-galactosyl transferase which is able to transfer the galactosyl residue of UDP-Gal to the N-acetyl-glucosaminylated lactose (lacto-N-triose II or LNT-2) forming LNT (lacto-N-tetraose) and genes encoding a biosynthetic pathway to UDP-GlcNAc, UDP-Gal. The fermentation was performed by culturing the strain in the presence of exogenously added lactose and a suitable carbon source, thereby producing LNT which was accompanied with lacto-N-triose II, para-LNH II and unreacted lactose as major carbohydrate impurities in the fermentation broth.

### Purification:

1. Ultrafiltration (UF)/Nanofiltration (NF): The obtained broth was acidified with H₂SO₄ to pH=3.8 and passed through continuous multi-loop industrial UF system equipped with 15 kDa ceramic membranes at T=60° C. with DF water flow of 2.5 and retentate flow 0.5 relative to the feed flow rate. The generated UF permeate was passed immediately through continuous multi-loop NF system equipped with Trisep UA60 membranes at T=15° C. and TMP=30 bar and DF flow rate of ca. 2.5 relative to feed (UF permeate) flow rate.
2. Strong acidic cation exchange resin: A sample of the obtained NF retentate (4.8 kg, Brix 22.1, conductivity 3.48 mS/cm, pH 3.92, Abs_400 2.6926 (path=1 cm), CI_400 121.8) was passed through Dowex-88 resin (H-form, BV=800 ml) followed by elution with water to give 5.57 kg of acidic effluent which was pH-adjusted with 4% NaOH-solution to 3.50 (Brix 18.2, conductivity 4.93 mS/cm, Abs_400 nm 1.027).
3. Active charcoal decolorization: The obtained solution (5.54 kg) was passed through granulated active charcoal CPG LF (90 g, 180 ml) packed in a XK16/100 column (ID=16 mm) at T=60° C. and at 2 bed volumes per hour flow rate (360 ml/h) followed by water elution. The obtained nearly colourless solution had the following parameters: m=5.49 kg, Brix 17.7, conductivity 5.01 mS/cm, pH 4.24, Abs_400 0.0130, CI_400 0.73.
4. NF/DF: The obtained solution (5.370 kg) was pH-adjusted to 3.05 with 0.6 ml 25% H₂SO₄ and subjected to constant volume diafiltration in MMS SW18 membrane filtration system equipped with 1812-size spiral wound Trisep UA60 membrane under the following conditions: cross-flow=400 l/h, TMP=30 bar, T=30-35° C. and DF water flow 4.6 l/h. The pH was adjusted with extra H₂SO₄ to 3.5 with H₂SO₄ after 10 1 and 20 1 of DF water was added. After adding a total of 25 1 of DF water, the retentate had the following parameters: Brix 18.4, conductivity 0.30 mS/cm, pH 3.60. It was further concentrated at TMP=39 bar to give a final NF retentate (2.94 kg, Brix 29.2, conductivity 0.283 mS/cm, pH 3.52, Abs_400 0.0207, CI_400 0.71).
5. Microfiltration and freeze-drying: The obtained NF retentate was pH-adjusted to 4.78 with 0.2 ml 50% NaOH-solution and passed through a PES 0.2 μm micro-filter to give a colourless solution (2.82 kg), which was freeze-dried to give 788 g of an amorphous white solid.

1. A method for recovery and purification of a neutral or sialylated human milk oligosaccharide (HMO) from a fermentation broth, comprising the steps of:
I. separating the fermentation broth to form a separated HMO-containing stream and a biomass waste stream;
IIa. purifying the separated HMO-containing stream by nanofiltration (NF) or nanofiltration/diafiltration (NF/DF);
IIb. an acidic cation exchange resin treatment then a nanofiltration step, wherein the nanofiltration membrane has a molecular weight cut-off (MWCO) of 500-3000 Da, the active (top) layer composed of polyamide, the membrane has a MgSO₄ rejection factor of about 50-90% and a NaCl rejection factor of not more than 50%, and the nanofiltration step is performed so that the pH is set below 5.0;
III. optionally concentrating the purified HMO-containing stream; and
IV. drying the purified HMO-containing stream to obtain a solidified neutral or sialylated HMO,
with optional active charcoal treatment.

2. The method according to claim 1, wherein step I) comprises at least one of ultrafiltration, microfiltration, and centrifugation.

3. The method according to claim 1, wherein the acidic cation exchange resin in step IIb) is a strongly acidic cation exchange resin, preferably a styrene-divinylbenzene cation exchange resin.

4. The method according to claim 3, wherein the resin is in H⁺-form.

5. The method according to claim 1, wherein the method comprises further purification of the HMO-containing stream by an active carbon treatment.

6. The method according to claim 1, wherein step IIa) or IIb) further comprises nanofiltration conducted in diafiltration mode.

7. The method according to claim 1, wherein step III) comprises evaporation, reverse-osmosis filtration, or a combination thereof.

8. The method according to claim 1, wherein step III) comprises concentration with a nanofiltration membrane, wherein the nanofiltration membrane has a molecular weight cut-off (MWCO) of 150-300 Da.

9. The method according to claim 1, wherein step III) is conducted and step IV) consists of spray-drying to obtain solidified HMO.

10. The method according to claim 1 comprising the following steps:
i. separating the fermentation broth to form a separated HMO-containing stream and a biomass waste stream by ultrafiltration;
ii. purifying the separated HMO-containing stream by combined nanofiltration and diafiltration, wherein the nanofiltration membrane is in the range of 500-3000 Da MWCO;
iii. purifying the HMO-containing stream by a strongly acidic cation exchange resin in H⁺-form;
iv. purifying the HMO-containing stream by a second nanofiltration step, wherein the nanofiltration membrane has a molecular weight cut-off (MWCO) of 500-3000 Da, the active (top) layer composed of piperazine-based polyamide, the membrane has a MgSO₄ rejection factor of about 50-90% and a NaCl rejection factor of not more than 50%, and the nanofiltration step is performed so that the pH is set below 5.0;
v. concentrating the purified HMO-containing stream by evaporation; and
vi. spray-drying the purified HMO-containing stream to obtain a solidified neutral or sialylated HMO;
optionally with active charcoal treatment after step ii), iii), iv) or v).

11. The method according to claim 1 comprising the following steps:
i. separating the fermentation broth to form a separated HMO-containing stream and a biomass waste stream by ultrafiltration;
ii. purifying the separated HMO-containing stream by combined nanofiltration and diafiltration, wherein the nanofiltration membrane is in the range of 500-3000 Da MWCO;
iii. purifying the nanofiltration retentate by a strongly acidic cation exchange resin in H⁺-form;
iv. purifying the resin eluate by a second nanofiltration step, preferably combined with diafiltration, wherein the nanofiltration membrane has a molecular weight cut-off (MWCO) of 500-3000 Da, the active (top) layer composed of piperazine-based polyamide, the membrane has a MgSO₄ rejection factor of about 50-90% and a NaCl rejection factor of not more than 50%, and the nanofiltration step is performed so that the pH is set below 5.0;
v. optionally concentrating the nanofiltration retentate by evaporation or reverse osmosis; and
vi. freeze-drying the nanofiltration retentate or the concentrate to obtain a solidified neutral or sialylated HMO;
optionally with active charcoal treatment between steps iii) and iv).

12. The method according to claim 10, wherein step iii) further comprises the addition of NaOH-solution to the resin eluate so that the pH is set to 3-5.

13. The method according to claim 1 which does not comprise a basic anion exchange resin treatment.

14. The method according to claim 13 which does not comprise electrodialysis.

15. The method according to claim 1, wherein the HMO is a neutral HMO.

16. The method according to claim 15, wherein the HMO is selected from the group consisting of: 2′-fucosyllactose, 3-fucosyllactose, 2′,3-difucosyllactose, lacto-N-triose II, lacto-N-tetraose, lacto-N-neotetraose, lacto-N-fucopentaose I, lacto-N-fucopentaose II, lacto-N-fucopentaose III, lacto-N-fucopentaose V, lacto-N-fucopentaose VI, lacto-N-difucohexaose I, lacto-N-difucohexaose II, lacto-N-difucohexaose III, 6′-galactosyllactose, 3′-galactosyllactose, lacto-N-hexaose and lacto-N-neohexaose.

17. The method according to 15, wherein the HMO is 2′-fucosyllactose, 3-fucosyllactose, 2′,3-difucosyllactose, lacto-N-triose II, lacto-N-tetraose, lacto-N-neotetraose or a lacto-N-fucopentaose, preferably 2′-fucosyllactose, LNT, LNnT or a lacto-N-fucopentaose.

18. The method according to claim 1, wherein the HMO is a sialylated HMO.

19. The method according to claim 1, wherein the pH is set between 3.0 and 4.5.

20. The method according to claim 10, wherein the pH is set between 3.0 and 4.5.
